(19) United States
(12) Patent Application Publication
(10) Pub. No.: US 2024/0382147 A1
(43) Pub. Date: Nov. 21, 2024

(54) Methods and Apparatuses for Determination of Hot Flashes

(71) Applicant: 1625986 ONTARIO LIMITED, MT BRYDGES (CA)

(72) Inventors: Geoff Taylor, Treasure Island, FL (US); Vitaliy Degtyaryov, Oakbank (CA)

(21) Appl. No.: 18/693,859

(22) PCT Filed: Aug. 15, 2022
(86) PCT No.: PCT/IB22/00464

§ 371 Date: Mar. 20, 2024

Related U.S. Application Data
(60) Provisional application No. 63/235,953, filed on Aug. 23, 2021.

(51) Int. Cl. A61B 5/00; A61B 5/01; A61B 5/0531

(52) U.S. Cl. CPC A61B 5/4306; A61B 5/01; A61B 5/0531; A61B 2560/0252; A61B 2562/0214

(57) ABSTRACT

The subject invention provides methods and apparatuses that determine or characterize a physiological condition known as a “hot flash” in a subject. Embodiments include interdigitated sensors and control systems that determine measures of a plurality of impedance, capacitance, AC resistance, phase, DC resistance, and temperature of the skin of a subject using a plurality of electrodes at a plurality of measurement times over a total measurement period. Embodiments determine a composite capacitance, a combined phase, or a combination thereof, and determine or characterize a hot flash therefrom.

### TECHNICAL FIELD

The subject invention relates to the determination of a physiological condition known as a “hot flash” in a subject.

### BACKGROUND ART

A hot flash is the sudden feeling of warmth in the upper body, which is usually most intense over the face, neck and chest. The individual's skin might redden, as if they're blushing. A hot flash can also cause severe sweating. If the subject loses too much body heat, they might feel chilled afterward. A hot flash is believed to be the result of vascular changes which permit rapid increased blood flow through vessels

Although other medical conditions can cause them, hot flashes most commonly are due to menopause—the time when a woman's menstrual periods become irregular and eventually stop. In fact, hot flashes are the most common symptom of the menopausal transition. The frequency and intensity of hot flashes vary among women. Hot flashes may be mild or so intense that they make a woman feel nauseated and they can disrupt daily activities. They can happen at any time of day or night. Nighttime hot flashes (night sweats) may wake the patient from sleep and can cause long-term sleep disruptions. Lack of sleep due to constant disturbances by recurring hot flashes contributes to extreme fatigue which can in turn cause physiologic, emotional and in some women dependency issues. Women who experience hot flashes for a prolonged period of time can become dependent on alcohol, sleeping pills and other drugs to try and get some relief. The etiology and mechanism of hot flashes remain not fully understood.

The U.S. Food and Drug Administration (FDA) has approved the use of paroxetine, a low-dose selective serotonin reuptake inhibitor (SSRI) antidepressant, to treat hot flashes. Researchers are studying the effectiveness of other antidepressants in this class. Women who use an antidepressant to help manage hot flashes generally take a lower dose than people who use the medication to treat depression. Side effects depend on the type of antidepressant taken and can include dizziness, headache, nausea, jitteriness, or drowsiness.

Some women may choose to take hormones to treat their hot flashes. A hormone is a chemical substance made by an organ like the thyroid gland or ovary. Hormone therapy steadies the levels of estrogen and progesterone in the body. It is a very effective treatment for hot flashes in women who are able to use it. There are risks associated with taking hormones, including increased risk of heart attack, stroke, blood clots, breast cancer, gallbladder disease, and dementia. Currently there are also many supplements and remedies on the market with claims that their products stop hot flashes.

Determination of the efficacy of such treatments is problematic since there is no standard method or apparatus that objectively determines the presence, duration, or severity of a hot flash. Consequently, there is no quantifiable, repeatable way to assess hot flashes, and thus no way to quantify the effectiveness of any therapy. An accurate, non invasive, continuous monitoring device that measures hot flashes is needed to prove or disprove these available remedies and to aid those who are studying novel ideas and products to cure hot flashes.

### DISCLOSURE OF INVENTION

Understanding of the present invention can be facilitated by the context of U.S. Provisional Patent Application 62/420,878, filed Nov. 11, 2016; PCT Application PCT CA2017/051353, filed Nov. 14, 2017; U.S. application Ser. No. 16/348,231, filed May 8, 2019, issued as U.S. Pat. No. 10,888,245 on Jan. 12, 2021; Canadian Patent 3,043,242, filed Nov. 14, 2017; U.S. Provisional Patent Application 62/864,827, filed Jun. 21, 2019; PCT Application 2020/050844, filed Jun. 18, 2020; U.S. application Ser. No. 17/258,520, filed Apr. 30, 2021; Canadian Patent 3,105,315, filed Jun. 18, 2020; U.S. Provisional Patent Application 63/152,964, filed Feb. 24, 2021, each of which is incorporated herein by reference.

Embodiments of the present invention comprise a plurality of interdigitated electrically conductive elements. The spacing between the conductive elements can be non-uniform in some embodiments. The elements can be implemented, as examples, as traces on a circuit board, conductors on a stick-on surface or patch, conductors on the inside of a wristband, conductors on the bottom of a watch, electrically conductive paint or tattoo, conductors embedded under the surface of the skin, electrically conductive threads in the fabric of an elastic band, etc.

The conductive elements can be coated or covered with an electrically insulating material to insulate from direct electrical contact with the skin. Reducing direct contact with the skin can reduce the effect of changes in surface conductance, e.g., due to sweat, contact force, intimacy of contact, or combinations thereof. They can be left uncoated to allow direct electrical contact with the skin for measurement modes that measure with more emphasis on resistance or conductance. In some embodiments, both coated and uncoated elements are used, which can facilitate differentiation of surface and various subsurface depth characteristics of the tissue.

The conductive elements can be connected as two electrodes. In some embodiments, subsets of the conductive elements are grouped such that multiple distinct electrodes are provided. These multiple electrodes can be energized in various combinations to allow, e.g., different electrode densities, different electrode separations, or other characteristics of the subsets to be used to provide measurements tailored to specific skin conditions such as depth from the surface. The physiological measures vary at different depths due to the depth-dependent structure of the dermis, epidermis, and other tissue.

An example embodiment of the present invention provides an apparatus for the determination of a physiological condition known as a hot flash, comprising: (a) an interdigitated sensor; (b) a control system configured to apply electrical signals to the interdigitated sensor and collect signals from the interdigitated sensor; and (c) an analysis system configured to accept signals from the interdigitated sensor, and to determine the presence, intensity, duration, or a combination thereof, of a hot flash from the interdigitated sensor signals.

An example embodiment further comprises a skin temperature sensor, and wherein the control system is further configured to collect signals from the skin temperature sensor, and wherein the analysis system is further configured to determine the presence, intensity, duration, or a combination thereof, of a hot flash from the interdigitated sensor signals and the skin temperature signals.

An example embodiment further comprises an ambient temperature sensor, and wherein the control system is further configured to collect signals from the ambient temperature sensor, and wherein the analysis system is further configured to determine the presence, intensity, duration, or a combination thereof, of a hot flash from the interdigitated sensor signals and the ambient temperature signals.

In an example embodiment, the interdigitated sensor comprises a plurality of electrical conductors spaced apart by predetermined distances.

In an example embodiment, the electrical conductors are coated with a dielectric that prevents direct electrical contact between the electrical conductors and the skin of a subject. In an example embodiment, the interdigitated sensor comprises three electrical contacts, each having a plurality of fingers disposed on ⅔ of the circumference of each of a plurality of concentric circles, where each electrical contact is centered the circumference of one of the circles and the centers of the electrical contacts are equally spaced about such circumference, and wherein the fingers of each electrical contact are interdigitated with the fingers of adjacent electrical contacts. In an example embodiment, the interdigitated sensor comprises first, second, and third electrical contacts, each comprising a plurality of fingers, wherein fingers of the first electrical contact are interdigitated with fingers of the second electrical contact and separated by a first distance, and fingers of the second contact are interdigitated with fingers of the third contact and separated by a second distance, where the second distance is different than the first distance. In an example embodiment, the interdigitated sensor comprises first and second electrical contacts, each contact having fingers disposed in a fractal pattern. In an example embodiment, the control system is configured to apply an electrical signal to the interdigitated sensor, wherein the electrical signal comprises an alternating voltage at a frequency from 5 kHz to 95 kHz. In an example embodiment, the analysis system is configured to determine the presence of a hot flash when the signals collected from the interdigitated sensor indicate a capacitance from 0.1 nF to 0.4 nF. In an example embodiment, the analysis system is configured to determine the presence of a hot flash when the signals collected from the interdigitated sensor indicate a decrease in capacitance from of 0.01 to 0.08 nF within 10 seconds. In an example embodiment, the analysis system is configured to determine the presence of a hot flash when the signals collected from the interdigitated sensor indicate a decrease in capacitance from of 0.03 to 0.05 nF within 5 seconds.

An example embodiment provides a method of determining the presence, intensity, duration, or a combination thereof, of a hot flash, comprising: (a) determining a measure of a plurality of impedance, capacitance, AC resistance, phase, DC resistance, and temperature of the skin of a subject using a plurality of electrodes at a plurality of measurement times over a total measurement period; (b) determining the presence, intensity, duration, or a combination thereof, of a hot flash from the measures determined in step (a).

In an example embodiment, step (a) comprises providing an apparatus as in claim 1, and using the apparatus to determine a measure of a plurality of impedance, capacitance, AC resistance, phase, DC resistance, and temperature of the skin of a subject using a plurality of electrodes at a plurality of measurement times over a total measurement period. In an example embodiment, step (a) comprises determining a composite capacitance, a combined phase, or a combination thereof. n an example embodiment, step (a) comprises applying an electrical signal to an interdigitated sensor, wherein the electrical signal comprises an alternating voltage at a frequency from 5 kHz to 95 kHz. In an example embodiment, step (b) comprises determining the presence of a hot flash when the signals collected from the interdigitated sensor indicate a capacitance from 0.1 nF to 0.4 nF. In an example embodiment, step (b) comprises determining the presence of a hot flash when the signals collected from the interdigitated sensor indicate a decrease in capacitance from of 0.01 to 0.08 nF within 10 seconds. In an example embodiment, step (b) comprises determining the presence of a hot flash when the signals collected from the interdigitated sensor indicate a decrease in capacitance from of 0.03 to 0.05 nF within 5 seconds.

### BRIEF DESCRIPTION OF DRAWINGS

FIG. 1 is a schematic illustration of an example embodiment.

FIG. 2 is a schematic illustration of an example embodiment.

FIG. 3 is a schematic illustration of an example embodiment.

FIG. 4 is a schematic illustration of an example embodiment.

FIG. 5 is a schematic illustration of an example embodiment.

FIG. 6 is a plot of composite capacitance dC over time obtained using an example embodiment.

FIG. 7 is a plot of measurement device temperature over time obtained using an example embodiment.

FIG. 8 is a plot of combined phase dPH over temperature obtained using an example embodiment.

FIG. 9 is a plot showing hot flash peaks obtained using an example embodiment.

FIG. 10 is an outline of steps in a computer software implementation of an example embodiment.

FIG. 11 is a block diagram of an example embodiment of the invention.

### MODES FOR CARRYING OUT THE INVENTION AND INDUSTRIAL APPLICABILITY

### Example Embodiment

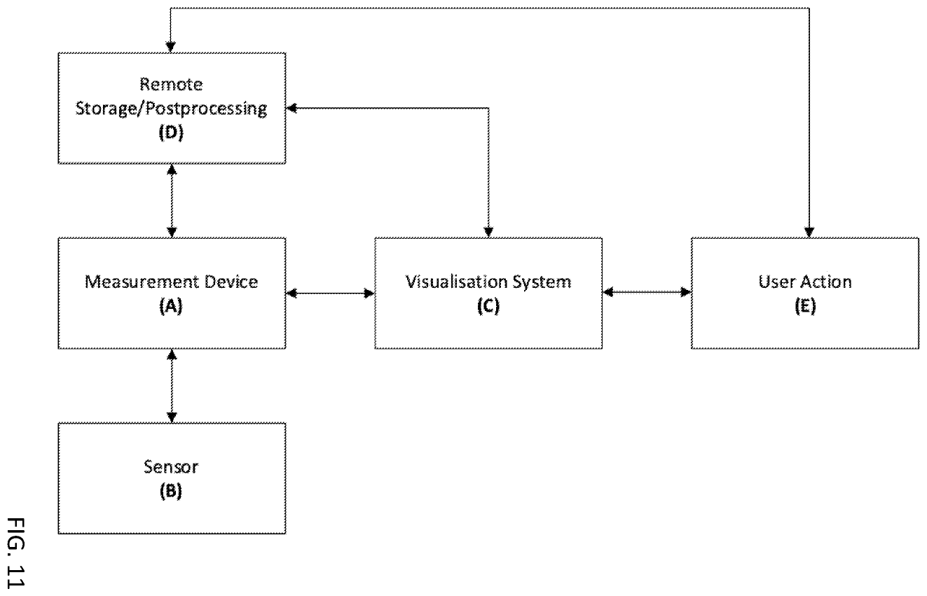

FIG. 11 is a block diagram of an example embodiment of the invention. The example embodiment comprises a Measurement Device, a sensor, a Visualization System, and a Remote Storage/Processing System. The Sensor (B) is attached to or placed in close proximity to the skin of a user and converts the user's physiological processes into electrical signals. The Measurement Device (A) converts electrical signals from the Sensor (A) into digital data for further storage and postprocessing. The Measurement Device implements algorithms for immediate processing of data and presentation of results to the user in real-time or on-demand from the user. Visualization of results is presented in a human readable format by the Visualization System (C). The Measurement System uses the Remote Storage/Postprocessing System (D) for storing history of measurements, remote access to measurements, and the advanced post-processing of user's data.

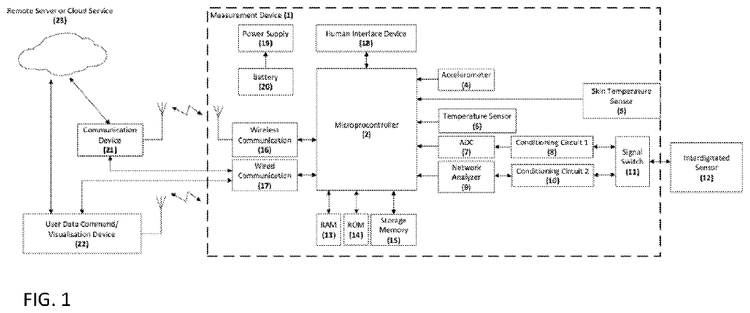

Example Embodiment. FIG. 1 is a schematic illustration of an example embodiment of the invention.

Measurement Device. The Measurement Device in the figure is a wearable device that generates and sends excitation signals to the Interdigitated Sensor (12) in the orderly manner, receives response signals from the Interdigitated Sensor (12), converts received signals into data and saves it into the internal memory. The measurement data is then processed by the Device for identification of hot flash events and displaying relevant results in real time or upon user's request by using embedded display or by sending data to other devices for visualization.

The Measurement Device has communication devices which allow to communicate either through wired communication interfaces or wireless interfaces to external devices with the following characteristics: processing of measured data; visualization of hot flash events; summarize parameters and provide statistical data for measured hot flashes over extended period of time; storing measured data; configuration of the Measurement Device; ease of access to measured data and results from remote locations.

The Measurement Device has a rechargeable battery and is capable of continuous measurements of hot flashes with pre-determined sampling frequency. The Measurement Device can be connected to the Internet directly, through Communication Device (21) or a User Data Command/Visualization Device (22).

Interdigitated Sensor. The Interdigitated Sensor is an electrical circuit represented by multiple electrical conductors with pre-determined distance between each other. Neighboring electrical conductors form pairs which are excited by the Measurement Device (1). Neighboring pairs of conductors are adjacent to each other, but not in electrical contact with each other such that electrical current can flow between them. The interdigitated side of the Sensor can come into a direct contact with the skin. The skin presents an externally complex impedance which is being excited with a known frequency and amplitude by the Measurement Device (1) or by the DC signal of known amplitude and current. The response signal on the Sensor is then measured and converted into digital data by the Measurement Device (1). Electrodes of the Interdigitated Sensor can be fully exposed and come into direct contact with the skin or they can be coated with a dielectric material of known properties, e.g., approximately a 0.001-to 0.005-inch-thick dielectric coating.

The Interdigitated sensor can be a rigid or a flexible printed circuit board with four pairs (or other number of pairs) of electrodes designed in such a way that each pair creates electrical circuits with unique electrical properties. Electrical properties of each electrode pair can be controlled by the following mechanical properties: space between electrodes; thickness of electrodes; total area of electrode pair.

Selection of orientation, thickness, area and space between electrodes can be dependent on the intended application. Energy density can be managed as a function of the sensor area and the applied voltage. Higher energies and larger spaces penetrate into deeper tissue and the returning signal is influenced by the deep tissue properties. Closer spaces and lower energies reflect the tissue properties nearer the surface. The following figures demonstrate various designs of interdigitated sensors used for taking measurements. The sensor design not limited to the shown examples; those skilled in the art will appreciate other designs and configurations contemplated by the invention and apparent from the present disclosure.

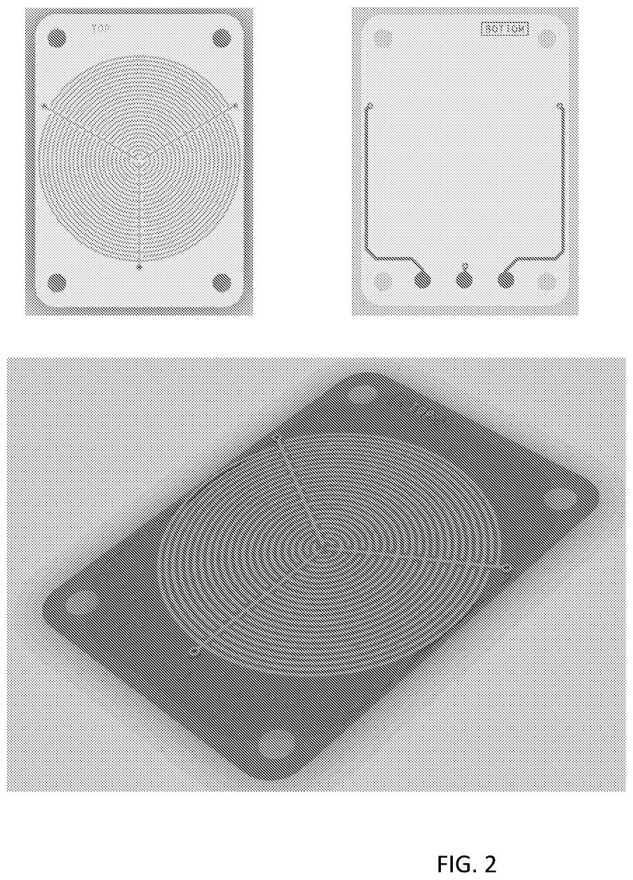

FIG. 2 is a schematic illustration of an example embodiment of an interdigitated sensor. The top left of the figure shows a top side view. The top right of the figure shows a bottom side view. The bottom of the figure shows an isometric top side view, with top and bottom copper layers shown. The example embodiment has three electrode pairs, with consistent 0.010″ spacing between all electrodes.

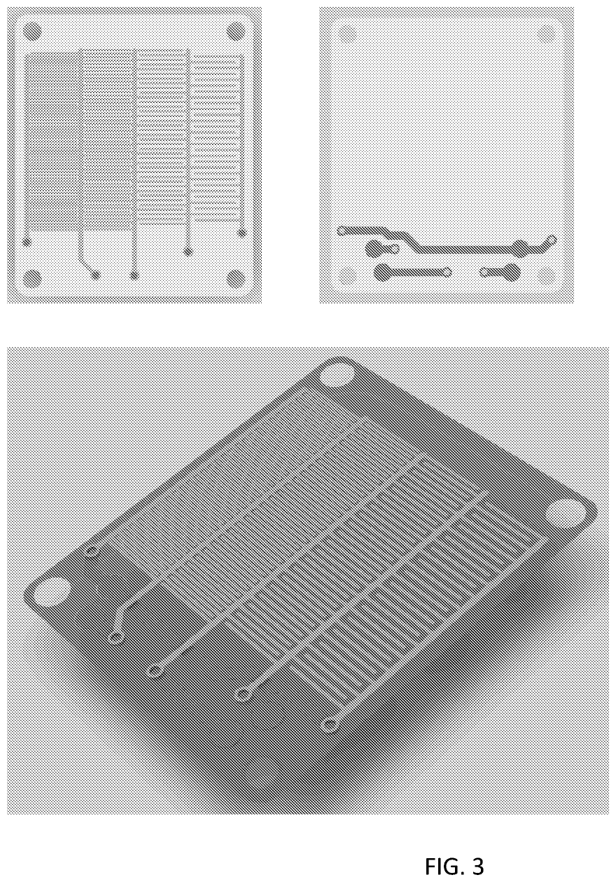

FIG. 3 is a schematic illustration of an example embodiment. The top left of the figure shows a top side view. The top right of the figure shows a bottom side view. The bottom of the figure shows an isometric top side view, with top and bottom copper layers shown. The example embodiment has four electrode pairs, with spacing (from left to right) 0.003″, 0.005″, 0.010″, 0.015″. All electrode pairs have the same thickness and different area.

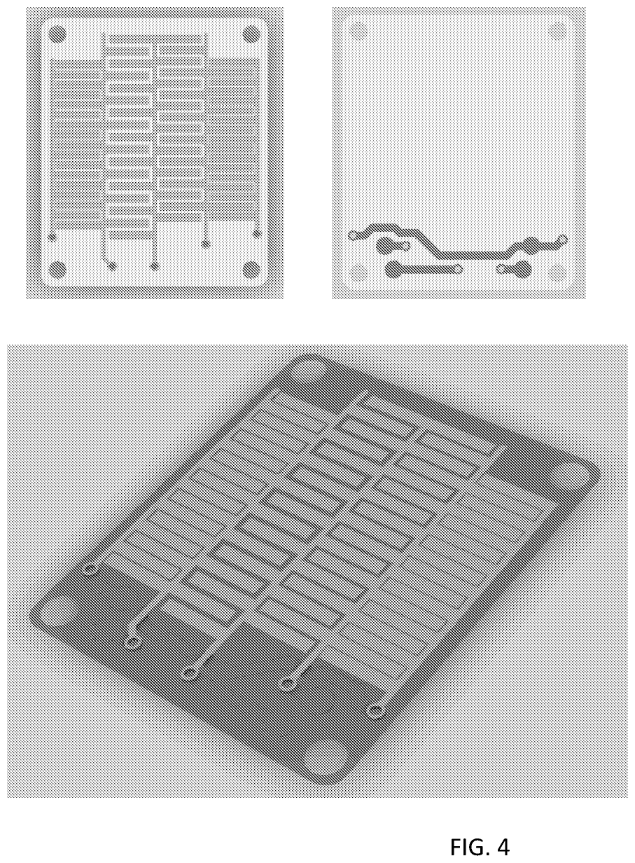

FIG. 4 is a schematic illustration of an example embodiment of an interdigitated sensor. The top left of the figure shows a top side view. The top right of the figure shows a bottom side view. The bottom of the figure shows an isometric top side view, with top and bottom copper layers shown. The example embodiment has four electrode pairs, with spacing (from left to right) 0.005″, 0.015″, 0.010″, 0.003″. All electrode pairs have the same thickness and the same area.

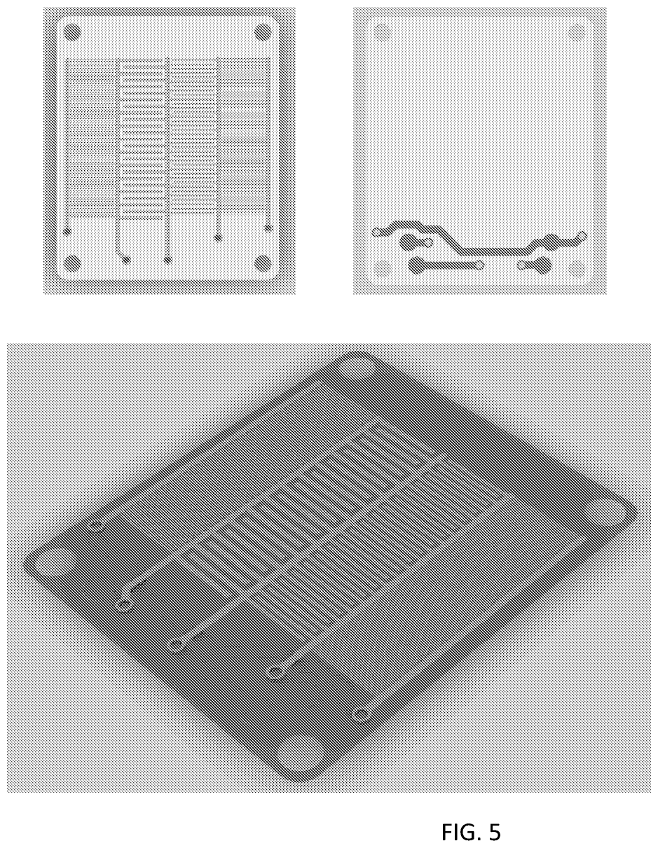

FIG. 5 is a schematic illustration of an example embodiment of an interdigitated sensor. The top left of the figure shows a top side view. The top right of the figure shows a bottom side view. The bottom of the figure shows an isometric top side view, with top and bottom copper layers shown. The example embodiment has four electrode pairs, with spacing (from left to right) 0.005″, 0.015″, 0.010″, 0.003″. Each electrode pair thickness is equal to the space between them. Electrode area for each electrode pair is equal.

In another configuration a pair of interdigitated traces can comprise a fractal pattern.

Communication Device. Communication Device is a device which has connection to the Internet. In case the Measurement Device (1) doesn't have a direct access to Internet, the Communication Device connects the Measurement Device to the Internet. The Communication Device uses wired or wireless interfaces to establish connection with the Measurement Device. Example communication devices include contemporary computers and modems.

User Data Command/Visualization Device. The User Data Command/Visualization Device can be used for the following: Configuration of the Measurement Device and its measurement circuits; Real-time processing of measurement data; Post-processing of measurement data; Real-time signaling of incoming hot flashes; Visualization of measurements; Storage of measurement data; Access to Cloud Service (23).

Examples of the User Data Command/Visualization Device include Computer, tablet, smart phone, smart watch, any device that has a display, storage memory, applicable wireless or wired interface for communication with the Measured Device and the Internet and necessary processing power for the tasks listed above.

Remote Server or Cloud Service. The Measurement system is using a Remote Server or a Cloud Service for the following: Storage of measurement data and for users; Remote access to measurement data; Remote access to results of measurements; Centralized processing of measurement data and delivery of results. Examples of Cloud Services include Amazon Web Services (AWS), Microsoft Cloud (Azure), Dropbox, Next Cloud. Examples of Remote Servers include computers which run server operating systems and capable for storage, processing and remote access to measurement data and results.

The Measurement Device (1) comprises the devices described below.

Microcontroller (2). Executes a software code which controls operation of the microcontroller; Collects data from sensors; Processes collected data in real-time or on-demand; Interfaces with attached hardware devices.

Human Interface Device (18). Displays measured and processed data; Interacts with a user to enter user selected data and configuration data into the Microcontroller. Examples: touch screen, displays of various technologies: LCD, OMOLED, TFT Display.

RAM (13) stores the software code, variables, measurement data.

ROM (14) stores the software code, variables and measurement data.

Storage Memory (15) stores measurement data, settings and configuration data for extended time.

Accelerometer (4) measures g-force in three directions X, Y, Z and is used for motion detection of the Measurement Device.

Skin Temperature Sensor (5) has a direct contact to the skin and measures skin temperature.

Temperature Sensor (6) is located inside the Measurement Device and measures internal device temperature.

ADC (7) measures resistance of skin between interdigitated electrodes.

Conditioning Circuit 1 (8) provides required signal amplitude and current to the interdigitated electrodes.

Network Analyzer (9) has the following functions: Generates a sequence of excitation signals with pre-set frequencies and amplitude; Receives the response signals from the Interdigitated Sensor; Processes the received signals in real-time and calculates impedance measured between interdigitated electrodes; Sends results of measurement to the Microcontroller.

Conditioning Circuit 2 (10) has the following functions: Scales and biases the excitation signals to the Interdigitate Sensor; Amplifies the response signals from the Interdigitated Sensor.

Signal Switch (11) Sequences through selected pairs of electrodes to allow one active electrode pair which performs measurement at a time.

Wireless Communication Device (16) uses wireless electromagnetic signals to communicate with external devices for the following: Transmission of measured data and results to the Internet, Communication Device (21) or the User Data Command/Visualization Device (22); Configuration of settings of the Measurement Device from the external device; Update of the software code of the Measurement Device from the external device; Transmission of user defined alarm signals from the Measurement Device to the external device. Examples include Bluetooth, WiFi, Zigbee, or other custom wireless devices and protocols.

Wired Communication (17) provides the same functionality as the Wireless Device over wired connection to the Communication Device (21) or the User Data Command/Visualization Device (22). It can also be used to charge the Battery (20). Examples include USB, RS232, 12C, SPI.

Battery (20) provides energy for autonomous operation of the Measurement Device.

Power Supply (19) converts energy stored in the Battery to the voltage levels used in the Measurement Device.

### Method of Measurement of Hot Flashes.

The description assumes a measurement device like the example described above. Other measurement devices can be used, and the method adjusted as required by the characteristics of the measurement device used.

The sensor is placed on the wrist or any other part of the body with electrodes towards the skin. The Measurement Device generates a set of excitation frequencies from f1 to fN. These signals then sent to each electrode pair of the Interdigitated Sensor in a sequence: space 1 to space N, one at a time. The excitation signals get modified by the combination of the electrical properties of the electrode pair and the skin impedance under the activated electrode pair. The response signal gets measured by the Measurement Device and compared to the generated excitation signal. Based on these differences, the Measurement Device calculates the impedance of the skin under each activated electrode pair for each generated frequency. Then, the Measurement Device calculates AC resistance, capacitance and phase of the signal based on the measured impedance for each generated frequency for each electrode pair of the Interdigitated Sensor.

Similarly, the Measurement Device sends the DC signal of a known amplitude to each electrode pair of the Interdigitated Sensor one at the time. The amplitude of this signal gets modified by combination of electrical properties of the electrode pair and the skin under activated electrodes at various depths as a function of the spacing of the electrodes. The Measurement Device measures the difference between the signal sent to each electrode pair and the measured signal from each pair and then calculates the DC resistance of skin.

Each sample of measured data contains measured impedance, capacitance, AC resistance, phase, DC resistance, and temperature data. Samples get collected periodically at a sample frequency sufficient to capture the hot flash, and for long time duration, e.g., overnight. This allows the system to obtain continuous measurements of the electrical properties of skin. Analysis of continuously measured electrical properties of skin at various frequencies and the input DC voltage allows to identify physiological conditions of the person who is wearing the Measurement Device.

Combination of the measurement method and the methodology for identification of a physiological condition, known as hot flashes, of the person who is wearing the Measurement Device is not previously known in the art.

### Identification of Hot Flash.

The following is a demonstration of the proposed method for identification of hot flashes. The data samples were collected continuously each 4 minutes for two days. The Measurement Device uses the coated sensor shown in FIG. 4.

For isolation of hot flashes from the rest of signals, the composite capacitance dC is calculated as:

$$dC = {\sum }_{i = 1}^{N} {\sum }_{j = 1}^{M} {C}_{ij}$$

### Where:

i—frequency index, [1 . . . N]
j—space index of the Interdigitated Sensor, [1 . . . M]
C_ij—measured skin capacitance at frequency i under electrode pair j
N—Total number of frequencies in a sweep for each electrode pair
M—Total number of electrode pairs in the Interdigitated Sensor.

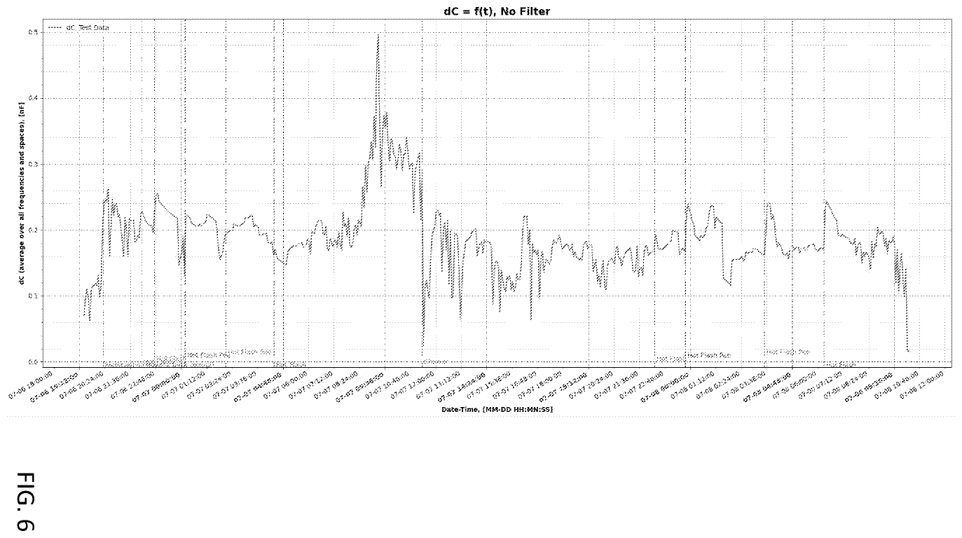

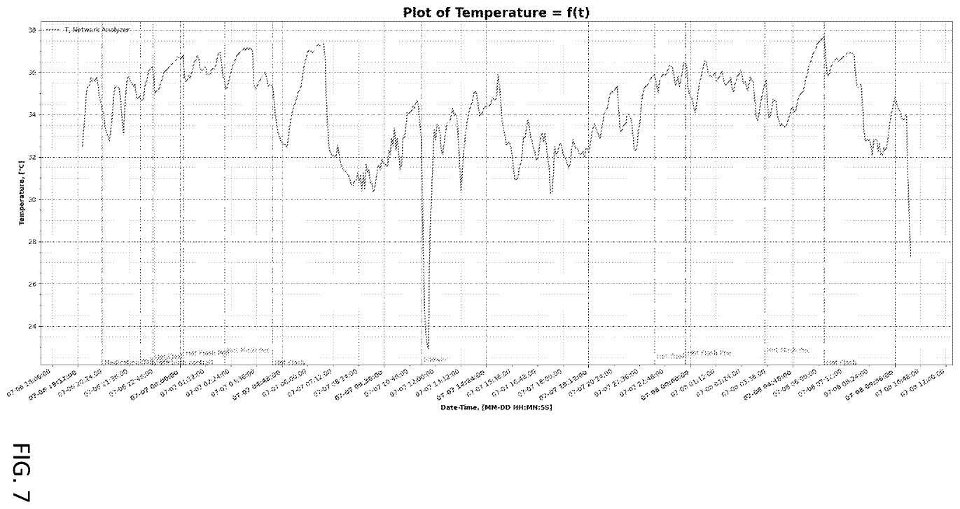

Plot of the composite capacitance dC calculated for 10 frequencies from 5 kHz to 95 kHz with frequency step 5 kHz is shown in FIG. 6 (Plot of composite capacitance dC over time). Comparison of this plot with the plot of measured temperature shown in FIG. 7 (Plot of Measurement Device's temperature over time) shows that the hot flash events start when the dC starts rising and the temperature drops at the same time.

Similar behavior can be observed when we use the combined phase dPH calculated as follows:

$$dPH = {\sum }_{i = 1}^{N} {\sum }_{j = 1}^{M} {PH}_{ij}$$

### Where:

i—frequency index, [1 . . . N]
j—space index of the Interdigitated Sensor, [1 . . . M]
[(PH)]_ij—measured phase of the skin impedance at frequency i under electrode pair j
N—Total number of frequencies in a sweep for each electrode pair
M—Total number of electrode pairs in the Interdigitated Sensor.

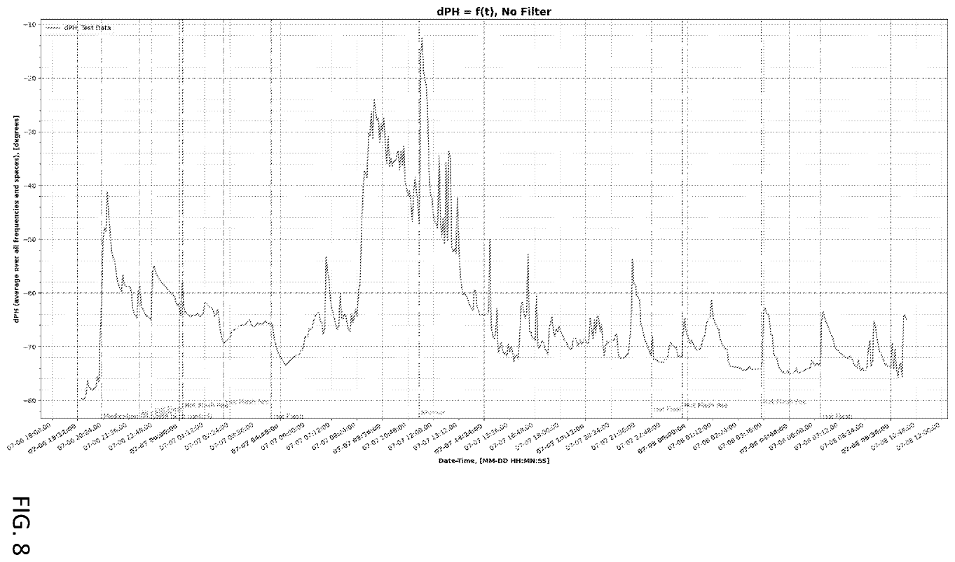

Comparison of dC and dPH show that dC tracks hot flashes better than dPH in this example embodiment. FIG. 8 provides a plot of combined phase dPH over temperature.

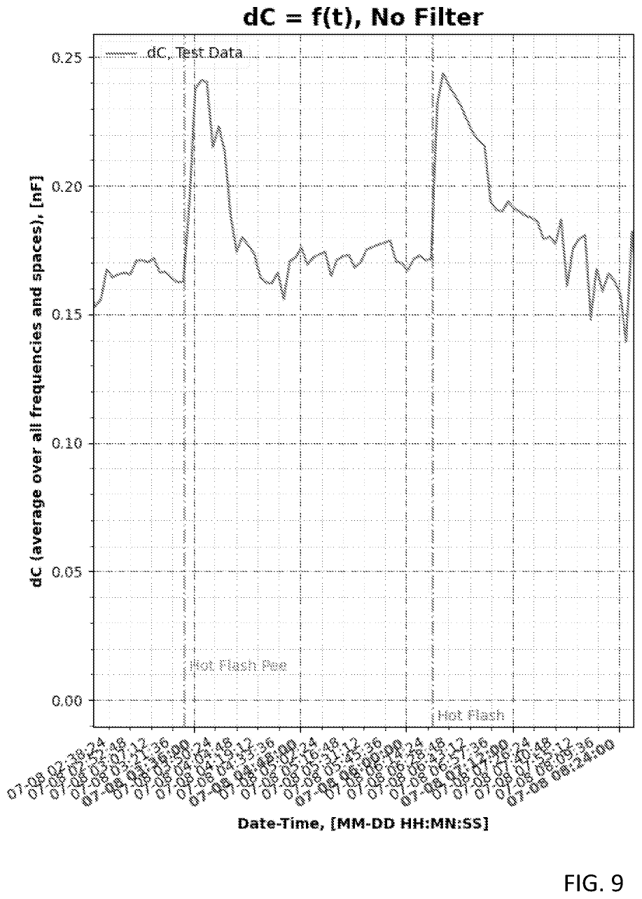

Based on the shape of the dC and the temperature curves and the notes of the person in the experiment, duration of the hot flash can be estimated as a duration of the peaks of the dC signal in the hot flash event as shown in FIG. 9 (Hot flash peaks).

Severity of the hot flash can be estimated by the amplitude, duration and the rising edge on the dC plot in combination with the amplitude of the temperature drop on the temperature plot. It was observed, the person feels the hot flashes the most at the peaks of the dC hot flash events. Note that temperature is not required in the determination of the initiation, intensity or duration of the hot flash. Temperature is an indicator that the body is responding to the hormones that triggered the hot flash and a reflection of the body's attempt to rectify situation. Temperature is influenced by clothing, airflow etc., whereas the tissue properties are not.

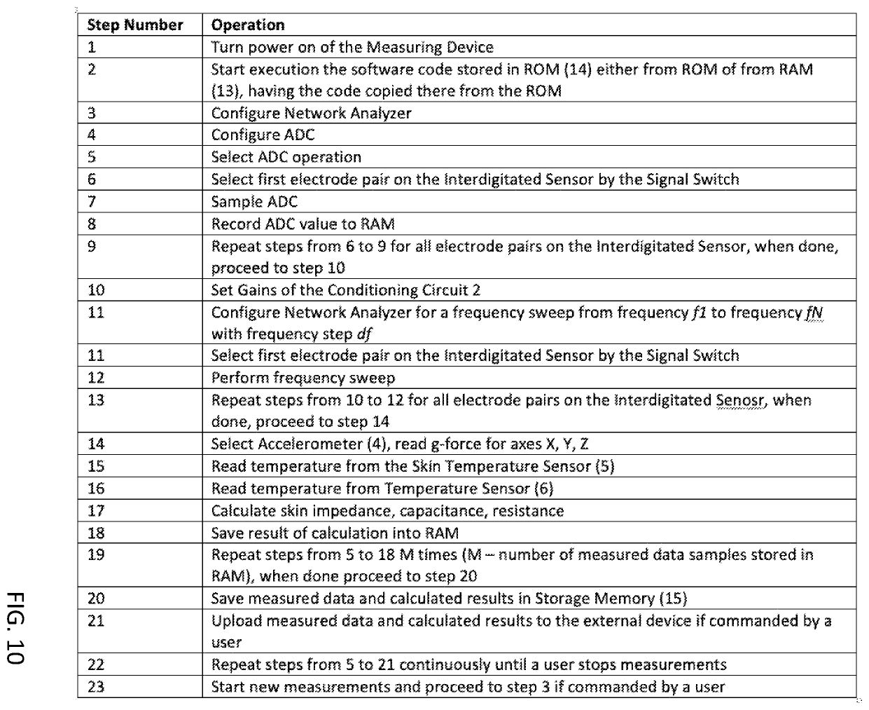

Software Implementation Example. An example method according to the present invention can be implemented in computer software guided by the outline presented in FIG. 10.

Continuous Sensing. Example embodiments can provide for multiple measurements over a time period, where the time period can be set as predetermined start and stop times, predetermined duration after a start signal or event, beginning at a start signal or event and extending until a stop signal or event, or continuous until stopped, as examples. Measurements over an extended period of time can allow determination of hot flashes under various conditions, e.g., day and night, awake and sleeping, etc.; determination and comparison of the parameters of individual hot flash events; timing and frequency of hot flash events; number of hot flash events; grouping of hot flash events around particular times, events, or conditions; periodicity and repeatability of individual hot flashes or groups of hot flashes around particular times, events, or conditions; and correlation of hot flash events with particular events or conditions, as examples.

### Applications.

Embodiments of the invention can provide information related to hot flashes, including, as examples, advance notice of an impending hot flash, verification of the occurrence of a hot flash, and indication of the relative severity of a hot flash. Such information can be communicated to the user, stored for future reference and comparisons, and communicated to others such as doctors, partners, family members, advocates, caregivers, researchers, clinical researchers, friends and support organizations. Embodiments of the invention can provide information useful for support of the user; knowledge of the cause and effect of hot flashes; increasing awareness of hot flash by the public, society, medical, and government; and to facilitate cures, trials, and remedies for hot flashes. We have noticed that the metabolic physiological changes occur and can be measured just prior to when the subject notices that they are having a hot flash and this can be beneficial for medical interventions.

Measurements enabled by the invention, optionally coupled to communication as described above, can assist a patient in several way. As examples, such information can help the patient deal with anxiety, depression, suicidal thoughts and other mental health issues; addictions to alcohol, drugs, food and other substances in attempts to deal with hot flashes; dehydration and muscle cramping; weight gain; lack of sleep which in turn can lead to loss of the health benefits of sleep, immune dysregulation, increased inflammation, risk of type-2 diabetes; insulin resistant states; weakened immune system, and risk of obesity; cognitive impairment and poor judgement; and uncharacteristic disagreeableness and argumentative tendencies.

Embodiments of the invention can be included in trials and dosing schemes for applications such as trials of new drugs and new therapies; dosing of medications; evaluating the efficacy of non-prescription therapies such as nutraceuticals and supplements; monitoring overall fitness; and monitoring therapies such as psychiatric, meditation, yoga, group therapy sessions and various others.

Embodiments of the invention can communicate with, or control, environmental systems to alleviate symptoms of hot flashes. As examples: turn on cooling elements such as ceiling fans near the onset of a hot flash; adjust a room or house air conditioner; adjust a room or house furnace or other heater; adjust the temperature inside a vehicle; initiate a cooling system coupled with the subject's bed or side of a bed; initiate a “bed jet” system to cool the subject; inject or control the dosing of a drug; locally cool parts of the subject such as the wrist or neck; initiate or control a local, personal fan; do any such action in anticipation of a hot flash if measurements allow anticipation of occurrences.

A subject can also use the objective measurements provided by embodiments of the invention to influence lifestyle changes; change personal habits, provide knowledge for emotional support and to facilitate other changes; and transmit measurements to medical personnel or other caregivers to facilitate effective therapy.

The present invention has been described in connection with various example embodiments. It will be understood that the above descriptions are merely illustrative of the applications of the principles of the present invention, the scope of which is to be determined by the claims viewed in light of the specification. Other variants and modifications of the invention will be apparent to those skilled in the art.

What is claimed is:

1. An apparatus for the determination of a physiological condition known as a hot flash, comprising:
(a) an interdigitated sensor;
(b) a control system configured to apply electrical signals to the interdigitated sensor and collect signals from the interdigitated sensor;
(c) an analysis system configured to accept signals from the interdigitated sensor, and to determine the presence, intensity, duration, or a combination thereof, of a hot flash from the interdigitated sensor signals.

2. The apparatus of claim 1, further comprising a skin temperature sensor, and wherein the control system is further configured to collect signals from the skin temperature sensor, and wherein the analysis system is further configured to determine the presence, intensity, duration, or a combination thereof, of a hot flash from the interdigitated sensor signals and the skin temperature signals.

3. The apparatus of claim 1, further comprising an ambient temperature sensor, and wherein the control system is further configured to collect signals from the ambient temperature sensor, and wherein the analysis system is further configured to determine the presence, intensity, duration, or a combination thereof, of a hot flash from the interdigitated sensor signals and the ambient temperature signals.

4. The apparatus of claim 1, wherein the interdigitated sensor comprises a plurality of electrical conductors spaced apart by predetermined distances.

5. The apparatus of claim 4, wherein the electrical conductors are coated with a dielectric that prevents direct electrical contact between the electrical conductors and the skin of a subject.

6. The apparatus of claim 4, wherein the interdigitated sensor comprises three electrical contacts, each having a plurality of fingers disposed on ⅔ of the circumference of each of a plurality of concentric circles, where each electrical contact is centered the circumference of one of the circles and the centers of the electrical contacts are equally spaced about such circumference, and wherein the fingers of each electrical contact are interdigitated with the fingers of adjacent electrical contacts.

7. The apparatus of claim 4, wherein the interdigitated sensor comprises first, second, and third electrical contacts, each comprising a plurality of fingers, wherein fingers of the first electrical contact are interdigitated with fingers of the second electrical contact and separated by a first distance, and fingers of the second contact are interdigitated with fingers of the third contact and separated by a second distance, where the second distance is different than the first distance.

8. The apparatus of claim 1, wherein the interdigitated sensor comprises first and second electrical contacts, each contact having fingers disposed in a fractal pattern.

9. The apparatus of claim 1, wherein the control system is configured to apply an electrical signal to the interdigitated sensor, wherein the electrical signal comprises an alternating voltage at a frequency from 5 kHz to 95 kHz.

10. The apparatus of claim 1, wherein the analysis system is configured to determine the presence of a hot flash when the signals collected from the interdigitated sensor indicate a capacitance from 0.1 nF to 0.4 nF.

11. The apparatus of claim 1, wherein the analysis system is configured to determine the presence of a hot flash when the signals collected from the interdigitated sensor indicate a decrease in capacitance from of 0.01 to 0.08 nF within 10 seconds.

12. The apparatus of claim 11, wherein the analysis system is configured to determine the presence of a hot flash when the signals collected from the interdigitated sensor indicate a decrease in capacitance from of 0.03 to 0.05 nF within 5 seconds.

13. A method of determining the presence, intensity, duration, or a combination thereof, of a hot flash, comprising:
(a) determining a measure of a plurality of impedance, capacitance, AC resistance, phase, DC resistance, and temperature of the skin of a subject using a plurality of electrodes at a plurality of measurement times over a total measurement period;
(b) determining the presence, intensity, duration, or a combination thereof, of a hot flash from the measures determined in step (a).

14. The method of claim 13, wherein step (a) comprises providing an apparatus as in claim 1, and using the apparatus to determine a measure of a plurality of impedance, capacitance, AC resistance, phase, DC resistance, and temperature of the skin of a subject using a plurality of electrodes at a plurality of measurement times over a total measurement period.

15. The method of claim 13, wherein step (a) comprises determining a composite capacitance, a combined phase, or a combination thereof.

16. The method of claim 13, wherein step (a) comprises applying an electrical signal to an interdigitated sensor, wherein the electrical signal comprises an alternating voltage at a frequency from 5 kHz to 95 KHz.

17. The method of claim 13, wherein step (b) comprises determining the presence of a hot flash when the signals collected from the interdigitated sensor indicate a capacitance from 0.1 nF to 0.4 nF.

18. The method of claim 13, wherein step (b) comprises determining the presence of a hot flash when the signals collected from the interdigitated sensor indicate a decrease in capacitance from of 0.01 to 0.08 nF within 10 seconds.

19. The method of claim 13, wherein step (b) comprises determining the presence of a hot flash when the signals collected from the interdigitated sensor indicate a decrease in capacitance from of 0.03 to 0.05 nF within 5 seconds.

20. The method of claim 13, wherein step (a) comprises determining a composite capacitance and a skin temperature, and wherein step (b) comprises determining a start of a hot flash when the composite capacitance increases at the same time as the skin temperature decreases.
